(19) United States
(12) Patent Application Publication
(10) Pub. No.: US 2008/0231809 A1
(43) Pub. Date: Sep. 25, 2008

(54) Method for Measuring Intraocular Lens

(76) Inventor: Wolfgang Haigis, Wurzburg (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/573,112

(22) PCT Filed: Sep. 18, 2004
(86) PCT No.: PCT/EP04/10506

§ 371 Date: Mar. 23, 2006

(30) Foreign Application Priority Data
Sep. 23, 2003 (DE) 103 44 781.4

(51) Int. Cl. A61B 3/107; A61F 2/16

(52) U.S. Cl. 351/246; 623/6.11

(57) ABSTRACT

The invention relates to a method for measuring an optimally adapted intraocular lens for patients having a refractively modified cornea. The inventive method includes the following steps: determining the formula-specific corneal refractive powers before the refractive intervention; determining the formula-specific corneal refractive powers after the refractive intervention, and; putting the formula-specific corneal refractive powers before and after the refractive intervention into the respective IOL formula.

The invention relates to a method for determining an intraocular lens (IOL) optimally adapted to the conditions in the patient's eye.

A current method, especially for treatment of cataract (lens opacification) is to remove the ocular lens (cataract surgery) and replace it by an artificial lens. This requires adaptation of the IOL refractive power P_{IOL} to the optical conditions so that the patient will regain full vision after the intervention.

The refractive power P_{IOL} of the intraocular lens depends on the one hand on the patient data to be collected (axis length L, corneal refractive power K, anterior chamber depth d, corneal radius R) and, on the other hand, on the characteristics of the intraocular lens to be implanted, expressed in the form of formula-specific lens constants (i.e. A constant, ACD constant, surgeon factor, pACD, a0, a1, a2 etc.).

P_{IOL}=f(L,K,d,R,A constant, . . . )

The respective patient's geometric values of axial length L, anterior chamber depth d and corneal radius R are measured using appropriate measuring instruments before surgery. A measuring instrument of that type is e.g. the IOL Master by Carl Zeiss Meditec.

The A constant depends on the IOL used, is determined by the IOL manufacturer and normally has a value between 118 and 119. The ACD constant describes the value of the anterior chamber depth adopted after surgery whereas the surgeon factor describes a correction factor which is doctor-specific. pACD is a personalized ACD constant, a0, a1 and a2 are specific empirically determined correction factors. A survey of these relations is given i.e. in the literature [1] Haigis W: Biometrie, in: Jahrbuch der Augenheilkunde 1995, Optik und Refraktion, Kampik A. (Ed.), Biermann-Verlag, Zülpich, 123-140, 1995, which is here fully referred to in content.

Different formulas have been developed for concrete calculation of the IOL parameters. According to the result of this calculation, an appropriate lens is selected from the IOL manufacturers' range and implanted in the patient.

American IOL formulas (SRK II, SRK/T, HofferQ, Holladay-1) expect the entry of the corneal refractive power in the form of a K value assuming that this K value is derived from the corneal anterior radius using a keratometer index of 1.3375. With normal (untreated) eyes, this corresponds to the entry of corneal back vertex power (D′C).

In addition, the K value or an intra-formula radius value derived for calculating the IOL position is applied.

Another formula is based on the inventor's findings (Haigis formula). For a better understanding of the invention, please refer to the following illustration:

(see source document)
mit=with
und=and
D: IOL refractive power
DC: corneal refractive power
RC: corneal radius
nC: (virtual) corneal refractive index nC=1.3315
ref: target refraction
dBC: vertex distance between cornea and glasses dBC=12 mm
d: optical anterior chamber depth
L: axis length (ultrasonically measured value)
n: refractive index of aqueous and vitreous (1.336)

The optical anterior chamber depth d is determined regressively from the preoperative ultrasonically measured values:

(see source document)
VKpr=preoperative anterior chamber depth (ultrasonically measured value)
ALpr=(=L) preoperative axis length (ultrasonically measured value)
MW(..)=average values for VKpr (=3.37) mm and ALpr (=23.39) mm
ACD-Konst:=ACD constant of the manufacturer

The relation between the ACD constant and the A constant specified by the manufacturer for intraocular lens characterization, results from:

A-Konst=(ACD-Konst+68.747)/0.62467

Whereas the constant a0 is related directly to the ACD constant of the manufacturer via (3), the following default values apply to a1 and a2: a1=0.4, a2=0.1 (see literature [1]). These parameters can be optimized by analyzing postoperative refraction data. Calculation is provided for each patient to determine the value d used to bring about the effectively postoperative refraction obtained from (1). The optical anterior chamber depths resulting are correlated with the preoperative ultrasonically measured values for anterior chamber and axial length according to (2). From this, the optimized constants a0, a1 and a2 directly result. These fit parameters are different for each lens so that they are suitable for characterizing a given intraocular lens.

All these formulas are adopted to normal eye conditions. Due to refractive procedures at the cornea to improve visual acuity (Photorefractive Keratectomy [PRK], Laser in Situ Keratomileusis [LASIK] etc.), these patients experience a change in their corneal refractive power which generally is reduced. The fundamental modification takes place in the anterior corneal surface, i.e. in the anterior surface refractive power. Depending on the procedure, the posterior surface is also affected. Both total and vertex refractive powers are changed by the intervention.

As a result, the effective anterior and posterior radii are required for exact calculation of the respective refractive powers.

However, these cannot be determined with sufficient accuracy when using common measuring instruments from opthalmologic practice.

In citation [2] N. Rosa, L. Capasso, A. Romano: A New Method of Calculating Intraocular Lens Power After Photoreactive Keratectomy, Journal of Refractive Surgery Vol 10, November/December 2002, p. 720 whose disclosures are hereby being fully referred to, these problems are explained in detail but without stating a satisfactory solution.

The invention is based on the assignment to overcome the disadvantages of prior art and to provide a method for calculating an optimally adapted IOL even in the event of modified corneal geometry due to refractive intervention.

According to the invention, this assignment can be solved by taking the steps listed in the main claim. A number of convenient extension studies are described in the dependent claims.

According to the invention, the method for IOL calculation after refractive corneal surgery consists of the following steps:
identification of the corneal refractive powers required for the respective IOL formula
measurement or derivation of the formula-specific corneal refractive powers (D12C_{prerf}, D′C_{preref}) before the refractive intervention
measurement or derivation of the formula-specific corneal refractive powers (D12C_{postref}, D′C_{postref}) after the refractive intervention
putting the formula-specific corneal refractive powers (D12C_{preref} and D12C_{postref} or D′C_{preref} and D′C_{postref}) before and after the refractive intervention into the respective IOL formula

For this purpose, the anterior and posterior corneal radii R1C_{preref}, R2C_{preref} before and R1C_{postref}, R2C_{postref} after the refractive intervention are determined.

For a better understanding of the invention, the geometrical conditions of the eye are specified according to the figures showing:

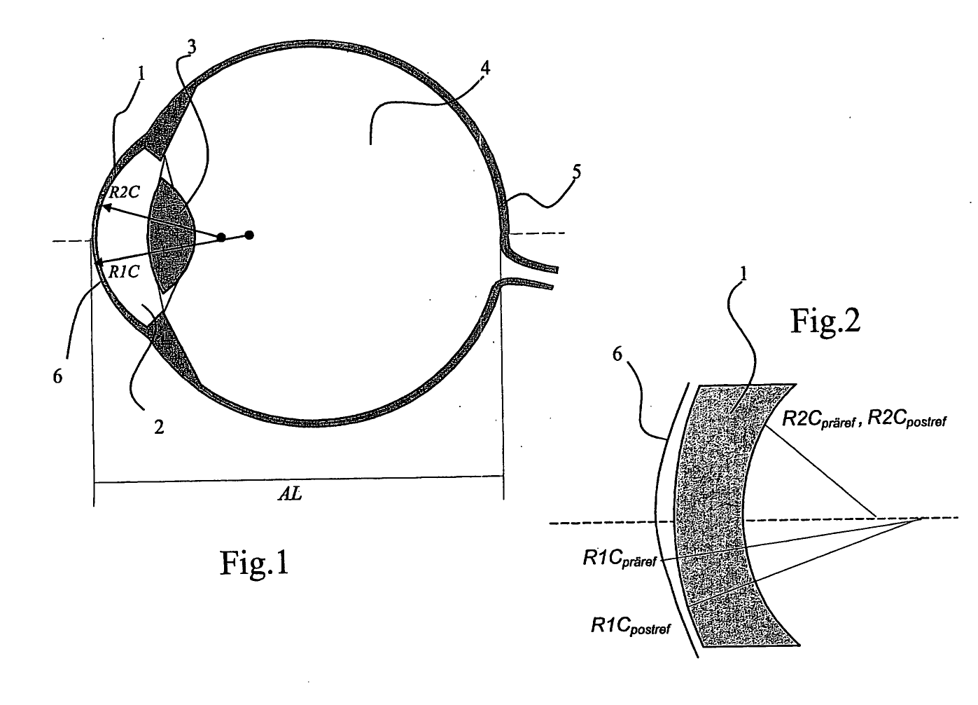

FIG. 1: a schematic cross-section of the eye

FIG. 2: a magnified detail of the cornea

In FIG. 1, the cross-section of the eye shows the cornea 1, anterior chamber 2, ocular lens 3, vitreous 4 and retina 5 with the corneal having an anterior radius R1C and a posterior radius of R2C. The distance between the corneal anterior surface 6 and the retina 6 is referred to as axial length AL. During cataract surgery, the ocular lens 3 is removed and replaced by an artificial intraocular lens.

FIG. 2 gives the geometrical conditions changed due to refractive surgery. A laser is used for targeted material ablation from the corneal anterior surface 6 or from the inner cornea after cornea dissection resulting in a different radius R1Cₚₒₛₜ instead of the preoperative radius R1Cₚᵣₑ. Due to modification of the corneal thickness, an altered corneal posterior radius R2C may result which, however, normally is far smaller than the changed anterior radius.

Apart from the refractive power of the ocular lens removed, the corneal refractive power also is to be considered when calculating the IOL.

The IOL is calculated according to the following scheme
R1C_{postref}, R2C_{postref}→refractive powers D12C_{postref}, D′C_{postref}
R1C_{preref}, R2C_{preref}→refractive powers D12C_{preref}, D′C_{preref}
putting D12C_{preref}, D12C_{postref} or D′C_{preref}, D′C_{postref} into the respective IOL formula

Both keratometry and topography have proved their worth for untreated eyes when calculating the corneal anterior radius.

In contrast, the measured values for common keratometry and topography of eyes after corneal refractive interventions are largely erroneous, especially for eyes after radial keratotomy as the radii determined are too steep. After PRK and LASIK treatments likewise, significant errors occur.

In case of eyes after refractive surgery the corneal anterior radius cannot be directly measured with sufficient accuracy. The other radii required are derived in an appropriate way.

Should patient data be unavailable before carrying out the refractive intervention, all radii must be derived.

Provided that keratometry is available before the refractive intervention, it is possible to derive the anterior radius effective after the intervention according to the “Refractive history method”, as described in the literature [3]: Haigis W: Homhautbrechkraft und Refraktionsmethode. Klin Monatsbl Augenheilk 220, Suppl 1, 17, 2003 which is here fully referred to in content.

When determining the different corneal radii required, the following cases can be distinguished:

### 1. Determination of R1C_{postref}

keratometry available before the refractive intervention (“LASIK pass”): derivation of R1C_{postref} from the ‘Refractive history method’
no data available before the refractive intervention:
measurement of R1C_{postref,apparent}
Transformation: R1C_{postref,apparent}=>R1C_{postref}
   R1C_{postref}=f1(R1C_{postref,apparent})

In this case, f1 is a device-specific transformation function which can be obtained by providing measuring instrument calibration. It is usually a regression line.

### 2. Determination of R1C_{reref}

keratometry available before the refractive intervention (“LASIK pass”):
derivation of R1Cₚᵣₑ from preoperative keratometry. This may require to consider the so-called keratometer index of the keratometer used.
no data available before the refractive intervention:
measurement of AL_{postref}
Transformation: AL_{postref}=>R1C_{preref}
   R1C_{preref}=f2(A_{Lpostref})

In this case, f2 is a transformation function which for instance has been determined statistically. In general, an S-shaped dependency of the corneal radius of the axis length can be expected here (R=R(AL)), as shown in the literature [4] Haigis W: Biometrie, in: Augenärztliche Untersuchungsmethoden, Straub W, Kroll P, Küchle HJ (Ed.), F.Enke Verlag Stuttgart, 255-304, 1995 whose disclosures are hereby being fully referred to.

The axial length available after the refractive intervention only slightly differs from the preoperative axial length (that is to say, by the ablation depth of typically approx. 150 μm) so that using the current postoperative axial length when deriving R1C_{pref} instead of the preoperative value of the axial length will produce negligible errors.

### 3. Determination of R2C_{preref}

previous measurement of R2C_{preref} (e.g. using an OrbScan II measuring instrument by Bausch & Lomb)
in case no measurement is possible:
   determination of R1C_{preref}
   transformation: R1C_{preref}=>R2C_{preref} R2C_{preref}=f3(R1C_{preref})

In this case, f3 is a transformation function for which e.g. the Gullstrand ratio g can be taken as a basis (R2C_{preref}=gR1C_{preref})

### 4. Determination of R2C_{postref}

measurement of R2C_{postref} (e.g. using OrbScan II)
in case no measurement is possible:
   determination of R2C_{preref}
   transformation: R2C_{preref}=>R2C_{postref} R2C_{postref}=f4(R2C_{preref})

In this case, f4 is a transformation function depending on the type of refractive intervention which in turn can be derived from the statistical evaluation of a sufficient number of patients. However, a good approximation is also provided by equating R2C_{postref}=R2C_{preref}, i.e. the influence of the refractive intervention on the corneal posterior radius R2C is disregarded.

The IOL is calculated using these refractive values and, if applicable, after conversion in the values required by the respective IOL formula.

The invention is not bound to the example presented. Further enhancements on a purely professional basis will not result in leaving the inventive method.

1-5. (canceled)

6. A method for determining an optimally adapted intraocular lens for patients having a refractively modified cornea, the cornea having been modified by a surgical refractive intervention, the method comprising:
determining pre-refractive intervention corneal refractive powers as required by a selected intraocular lens implant formula as they existed before the refractive intervention;
determining post-refractive intervention corneal refractive powers as required by the selected intraocular lens implant formula as they existed after the refractive intervention; and
utilizing determined values for pre-refractive intervention corneal refractive powers and post-refractive intervention corneal refractive powers to calculate the intraocular lens.

7. The method for determining an optimally adapted intraocular lens according to claim 6, wherein determining the corneal refractive powers before the refractive intervention comprises measuring a first anterior corneal radius and a first posterior corneal radius before the refractive intervention

8. The method for determining an optimally adapted intraocular lens according to claim 6, wherein determining the corneal refractive powers before the refractive intervention comprises deriving a first anterior corneal radius and a first posterior corneal radius before the refractive intervention from a second anterior corneal radius and a second posterior corneal radius measured after the refractive intervention.

9. The method for determining an optimally adapted intraocular lens according to claim 8, wherein derivation of the first anterior corneal radius and the first posterior corneal radius before the refractive intervention comprises transformation from the second anterior corneal radius and the second posterior corneal radius measured after the refractive intervention and wherein the transformation takes into account the parameters of the measuring instrument used for measuring the second anterior corneal radius and the second posterior corneal radius measured after the refractive intervention.

10. The method for determining an optimally adapted intraocular lens according to claim 9, wherein the parameters of the measuring instrument taken into account comprise a keratometer refractive index.

11. The method for determining an optimally adapted intraocular lens according to claim 8, wherein the determination of the first anterior corneal radius and the first posterior corneal radius before the refractive intervention from the second anterior corneal radius and the second posterior corneal radius measured after the refractive intervention comprises measuring to determine measured values and applying a correction value to the measured values.

12. The method for determining an optimally adapted intraocular lens according to claim 8, wherein the determination of the second anterior corneal radius and the second posterior corneal radius measured after the refractive intervention comprises derivation from the first anterior corneal radius and the first posterior corneal radius before the refractive intervention.

13. A method for determining an optimally adapted intraocular lens for patients having a refractively modified cornea, the cornea having been modified by a surgical refractive intervention, the method comprising:
selecting an intraocular lens implant formula;
determining pre-refractive intervention corneal refractive powers as they existed before the refractive intervention as required by the selected intraocular lens implant formula;
determining post-refractive intervention corneal refractive powers as they existed after the refractive intervention as required by the selected intraocular lens implant formula;
utilizing determined values for pre-refractive intervention corneal refractive powers and post-refractive intervention corneal refractive powers to calculate the intraocular lens via the selected intraocular lens implant formula.

14. The method for determining an optimally adapted intraocular lens according to claim 13, wherein determining the pre-refractive intervention corneal refractive powers comprises measuring a first anterior corneal radius and a first posterior corneal radius before the refractive intervention

15. The method for determining an optimally adapted intraocular lens according to claim 13, wherein determining the pre-refractive intervention corneal refractive powers
comprises deriving a first anterior corneal radius and a first posterior corneal radius before the refractive intervention from a second anterior corneal radius and a second posterior corneal radius measured after the refractive intervention.

16. The method for determining an optimally adapted intraocular lens according to claim 15, wherein derivation of the first anterior corneal radius and the first posterior corneal radius before the refractive intervention comprises transformation from the second anterior corneal radius and the second posterior corneal radius measured after the refractive intervention wherein the transformation takes into account the parameters of the measuring instrument used for measuring the second anterior corneal radius and the second posterior corneal radius measured after the refractive intervention.

17. The method for determining an optimally adapted intraocular lens according to claim 9, wherein the parameters of the measuring instrument taken into account comprise a keratometer refractive index.

18. The method for determining an optimally adapted intraocular lens according to claim 15, wherein the determination of the first anterior corneal radius and the first posterior corneal radius before the refractive intervention from the second anterior corneal radius and the second posterior corneal radius measured after the refractive intervention comprises measuring to determine measured values and applying a correction value to the measured values.

19. The method for determining an optimally adapted intraocular lens according to claim 8, wherein the determination of the second anterior corneal radius and the second posterior corneal radius measured after the refractive intervention comprises derivation from the first anterior corneal radius and the first posterior corneal radius before the refractive intervention.

20. The method for determining an optimally adapted intraocular lens according to claim 15, wherein derivation of the first anterior corneal radius and the first posterior corneal radius before the refractive intervention comprises transformation from the second anterior corneal radius and the second posterior corneal radius measured after the refractive intervention wherein the transformation takes into account the parameters of the measuring instrument used for measuring the second anterior corneal radius and the second posterior corneal radius measured after the refractive intervention.
